(19) United States
(12) Patent Application Publication
(10) Pub. No.: US 2013/0088358 A1
(43) Pub. Date: Apr. 11, 2013

(54) FASTENER TESTING DEVICE

(71) Applicants: FLY HE, JIANGSU PROVINCE (CN); CHING-FENG HSIEH, TAIPEI CITY (TW)

(72) Inventors: FLY HE, JIANGSU PROVINCE (CN); CHING-FENG HSIEH, TAIPEI CITY (TW)

(73) Assignees: ASKEY COMPUTER CORP.; ASKEY TECHNOLOGY (JIANGSU) LTD.

(21) Appl. No.: 13/324,085

(22) Filed: Dec. 13, 2011

(30) Foreign Application Priority Data
Oct. 7, 2011 (TW) 100136459

(51) Int. Cl. G08B 21/00; G01N 33/00

(52) U.S. Cl. 340/687; 73/865.8

(57) ABSTRACT

A fastener testing device includes a base, a testing unit, a mobile platform, and a prompting unit, tests an installation state between a plurality of installation holes on a subject and fasteners corresponding in position to the installation holes, respectively. The subject is mounted on the base. The testing unit is driven by the mobile platform to move close to the subject and test the installation state. The testing unit generates a test signal in response to a state of contact between the fasteners and probes corresponding in position to the installation holes, respectively. The prompting unit receives the test signal and informs each of the installation holes of a correct installation state or an incorrect installation state. Accordingly, incorrectly-installed ones of the installation holes are directly identified by the fastener testing device quickly, conveniently, and accurately.

### CROSS-REFERENCE TO RELATED APPLICATION

This non-provisional application claims priority under 35 U.S.C. §119(a) on Patent Application No(s).100136459 filed in Taiwan, R.O.C. on Oct. 7, 2011, the entire contents of which are hereby incorporated by reference.

### FIELD OF TECHNOLOGY

The present invention relates to fastener testing devices, and more particularly, to a fastener testing device for examining an installation state of engagement between installation holes of a subject and fasteners, respectively.

### BACKGROUND

A wide variety of electronic devices are each a subject composed of a circuit board, inner parts and components, and a housing. The subject is assembled by means of firm engagement between installation holes of the subject and fasteners, such as screws or bolts. To ensure product quality, it is necessary to perform a test for examining an installation state of engagement between the installation holes of the subject and the fasteners, respectively, after an installation process to preclude undesirable situations, such as a missing fastener or a loose fastener.

The aforesaid test is used to be carried out by visual inspection. However, the conventional test has its own drawbacks. It is time-consuming when there are plenty of installation holes on an electronic device and thus a testing worker has to inspect the installation holes one by one. It is difficult and laborious for the testing worker to inspect installation holes on different (for example, concave versus convex, or top versus bottom) surfaces of an electronic device. The fasteners inserted deep into the installation holes of a great depth are difficult to be visually inspected, thereby ending up with a high chance of carrying out a conventional visual inspection test imprecisely and allowing non-conforming products to pass the visual inspection test wrongly.

As a result, the conventional visual inspection test is prone to negligence and thus manmade errors, not to mention that it is time-consuming and labor-intensive, thereby incurring high costs.

### SUMMARY

It is an objective of the present invention to provide a fastener testing device for examining an installation state of engagement between installation holes of a subject and fasteners, respectively, in a quick, convenient, and precise manner.

Another objective of the present invention is to provide a fastener testing device for indicating how well each of the fasteners is installed in a corresponding one of the installation holes, so as to identify incorrect fastener-installed ones or empty ones of the installation holes of the subject.

Yet another objective of the present invention is to provide a fastener testing device for adjusting the manner of presentation of a testing unit to the height or position of each of the fasteners inserted into the installation holes of the subject, such that the test is conducted in a single process on the installation state of engagement between the installation holes and the fasteners, respectively.

In order to achieve the above and other objectives, the present invention provides a fastener testing device for examining an installation state of engagement between a plurality of installation holes of a subject and a plurality of fasteners, respectively, the fastener testing device comprising: a base defined with a receiving space thereabove for receiving the subject therein; a testing unit comprising a plurality of probes corresponding in position to the installation holes, respectively; a mobile platform disposed above the base and connected to the testing unit for driving the testing unit to move relative to the receiving space, such that the testing unit generates a test signal in response to a state of contact between each of the probes and a corresponding one of the fasteners; and a prompting unit electrically connected to the testing unit for generating prompting signals based on the test signals, respectively.

The prompting unit of the fastener testing device further comprises a plurality of light-emitting elements corresponding in position to the installation holes, respectively, for indicating an installation state of engagement between the installation holes and the fasteners therein, respectively. The light-emitting elements are light-emitting diodes.

The prompting unit of the fastener testing device further comprises an alerting unit for generating, based on the prompting signals, a correct installation state alerting signal for indicating a correct installation state and/or an incorrect installation state alerting signal for indicating an incorrect installation state. The alerting unit is a buzzer or a light alerting element. The light alerting element is a light-emitting diode.

The testing unit of the fastener testing device further comprises an adjusting portion for adjusting a manner of presentation of the probes to a shape of the subject or to a depth of the installation holes.

The probes of the fastener testing device each further comprise a triggering portion and a retractable probe. The triggering portion is electrically connected to the prompting unit and spaced apart from the retractable probe by a preset distance. After coming into contact with a corresponding one of the fasteners, the retractable probe travels the preset distance in the direction of the triggering portion to get electrically connected thereto, thus generating the test signal.

The mobile platform of the fastener testing device further comprises a handle, a connecting rod connected to the handle, and a lifting table connected to the connecting rod. The handle guides the connecting rod and drives the lifting table to ascend or descend. The testing unit is disposed beneath the lifting table.

The fastener testing device further comprises a mobile carrier for carrying the subject. The mobile carrier and the base have a guiding groove and a sliding rail, respectively, thereby allowing the mobile carrier to enter and exit the receiving space.

The fasteners and the installation holes are threaded correspondingly. For example, the fasteners are screwing elements, such as screws or bolts, and the installation holes are threaded to match the fasteners, respectively.

Accordingly, the fastener testing device of the present invention is used in examining an installation state of engagement between installation holes of a subject and fasteners, respectively, in a quick, convenient, and precise manner. The fastener testing device indicates how well each of the fasteners is installed in a corresponding one of the installation holes, so as to identify incorrect fastener-installed ones or empty ones of the installation holes of the subject. The fastener testing device generates an alerting signal for indicating that all the installation holes are correctly installed with the fasteners, respectively, and generates another alerting signal for indicating that one of the installation holes is empty or is not correctly installed with a corresponding one of the fasteners. The fastener testing device adjusts the manner of presentation of the testing unit to the height or position of each of the fasteners inserted into the installation holes of the subject, such that the installation state of engagement between the installation holes and the fasteners, respectively, can be examined in a single process. As a result, the fastener testing device of the present invention is not prone to negligence or manmade errors but is time-saving and labor-saving, thereby cutting costs.

### BRIEF DESCRIPTION

Objectives, features, and advantages of the present invention are hereunder illustrated with a specific embodiment in conjunction with the accompanying drawings, in which:

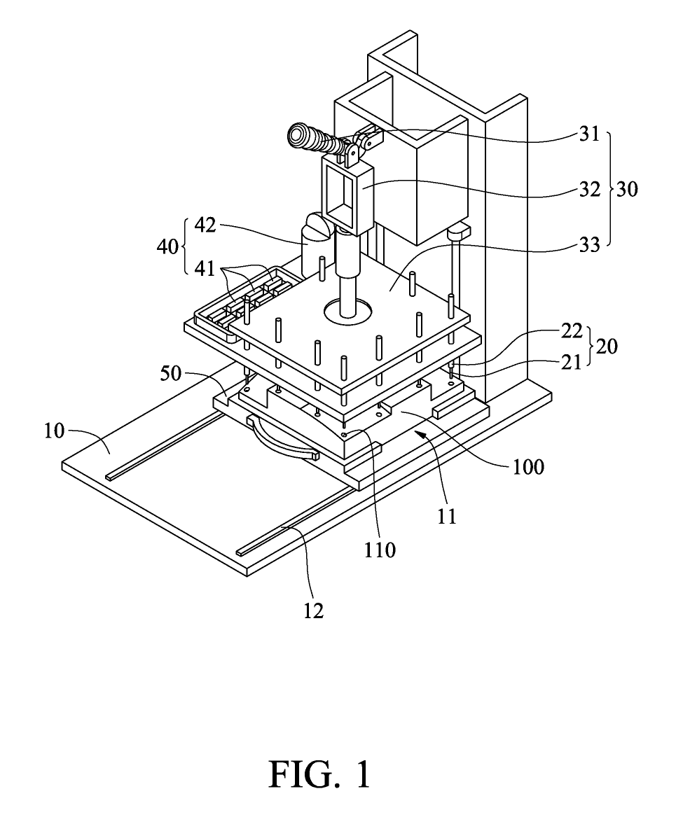

FIG. 1 is a schematic view of a fastener testing device according to the specific embodiment of the present invention;

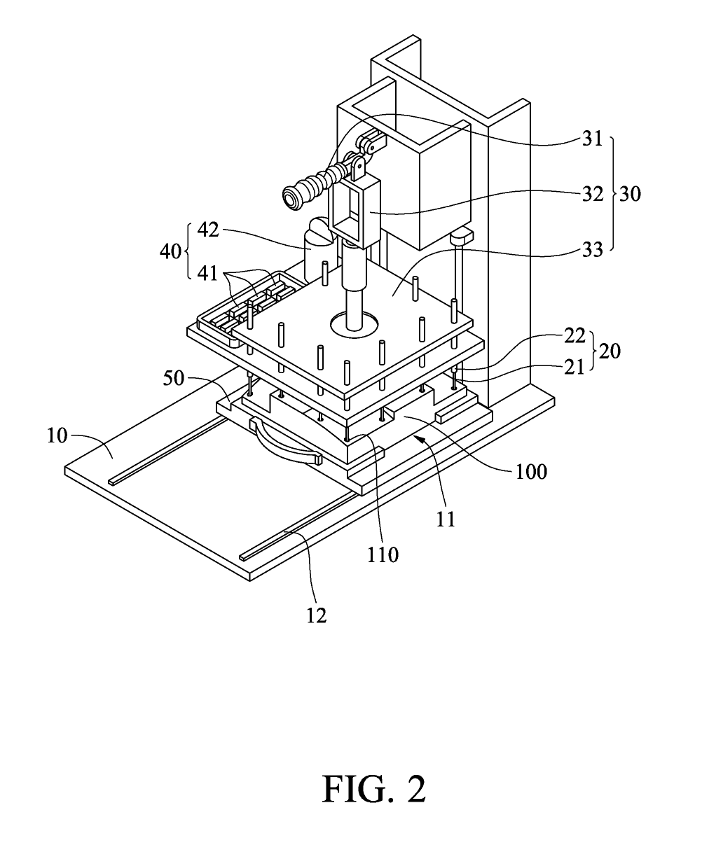

FIG. 2 is a schematic view of operating the fastener testing device in the specific embodiment of the present invention;

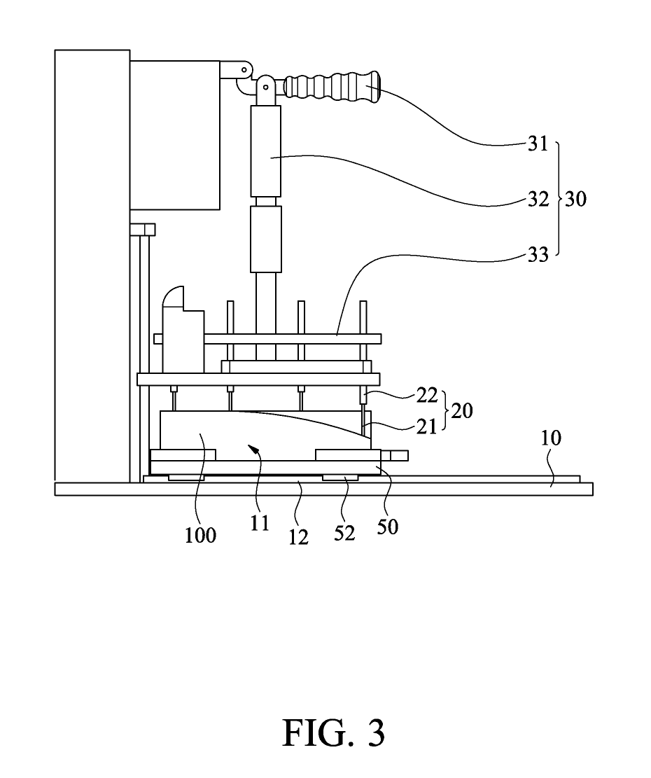

FIG. 3 is a lateral schematic view of operating the fastener testing device shown in FIG. 2;

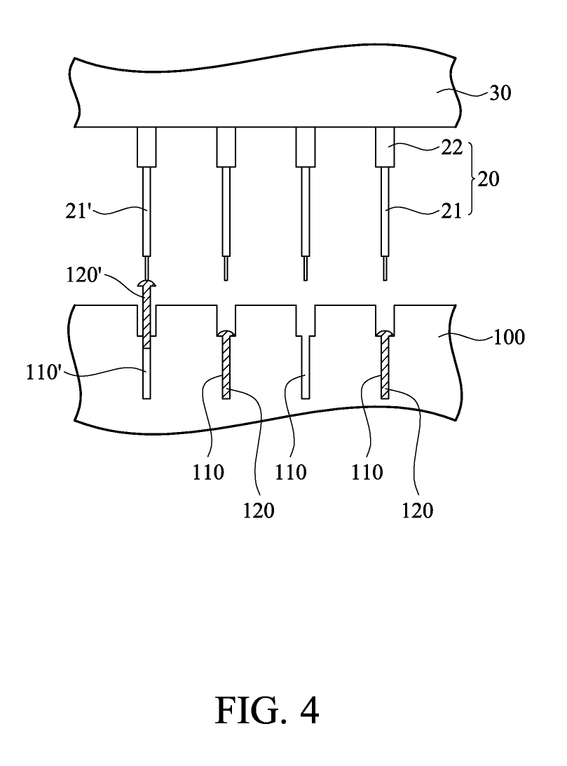

FIG. 4 is a schematic view of operating a plurality of probes of a testing unit of the fastener testing device in the specific embodiment of the present invention;

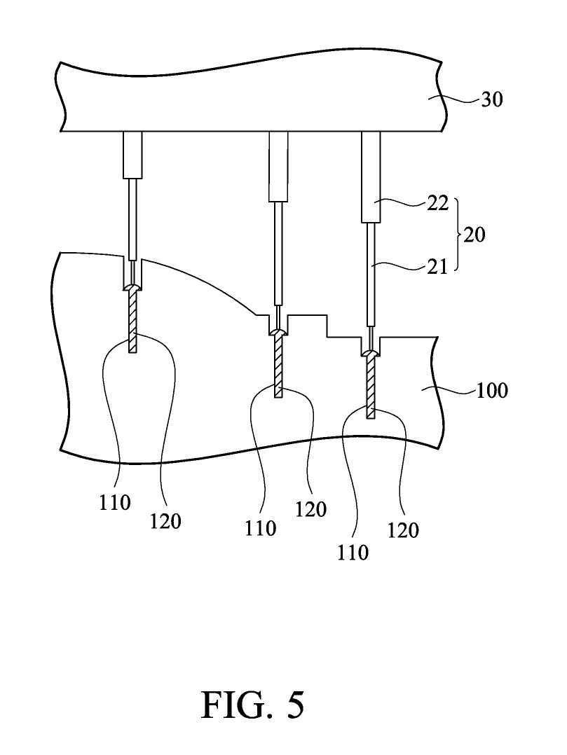

FIG. 5 is a schematic view of an adjusting portion of the testing unit of the fastener testing device in the specific embodiment of the present invention; and

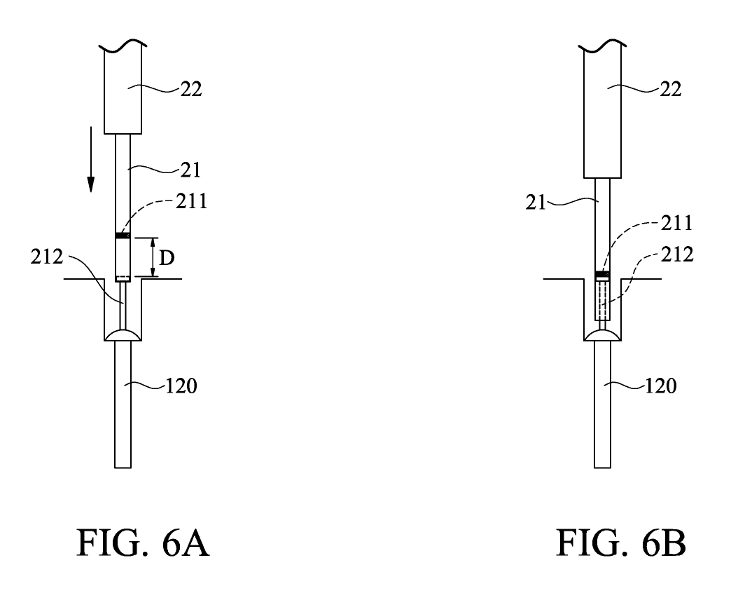

FIG. 6A and FIG. 6B are schematic views of the probes of the testing unit of the fastener testing device in the specific embodiment of the present invention.

### DETAILED DESCRIPTION

Referring to FIG. 1 through FIG. 3, there are shown schematic views of a fastener testing device according to the specific embodiment of the present invention. As shown in the drawings, the fastener testing device is provided and operated by a testing worker (not shown) to examine an installation state of engagement between a plurality of installation holes 110 of a subject 100 and a plurality of fasteners 120 corresponding in position thereto, respectively, so as to evaluate and determine whether the installation state is a correct installation state or an incorrect installation state. The correct installation state refers to the situation where all the fasteners 120 are correctly installed in all the installation holes 100, respectively. The incorrect installation state means that either at least one of the installation holes 100 is empty or at least one of the fasteners 120 is improperly installed in a corresponding one of the installation holes 100 (for example, incomplete engagement between the affected fastener 120 and the affected installation hole 100). In this embodiment, the fasteners 120 are exemplified by screws, and the installation holes 110 are threaded and thus engageable with the fasteners 120, respectively. However, the aforesaid disclosures in this embodiment are not restrictive of the fasteners 120; on the contrary, the fasteners 120 can come in the form of threaded bolts.

The fastener testing device in the specific embodiment of the present invention comprises a base 10, a testing unit 20, a mobile platform 30, and a prompting unit 40. The base 10 is defined with a receiving space 11 thereabove for receiving the subject 100 therein. The testing unit 20 is disposed above the receiving space 11 and comprises a plurality of probes 21 corresponding in position to the installation holes 110, respectively. The mobile platform 30 is disposed above the base 10 and connected to the testing unit 20 for driving the testing unit 20 to move relative to the receiving space 11, such that the testing unit 20 generates a test signal in response to a state of contact between each of the probes 21 and a corresponding one of the fasteners 120. The prompting unit 40 is mounted on and electrically connected to the testing unit 20 for generating prompting signals based on the test signals, respectively.

With the subject 100 received in the receiving space 11, the testing unit 20 driven by the mobile platform 30 to approach the subject 100 in the receiving space 11, and the probes 21 adapted to examine the engagement between the installation holes 110 and the fasteners 120 therein, respectively, the probes 21 come, in the presence an unconfirmed correct installation state of engagement between the installation holes 110 and the fasteners 120 therein, respectively, into contact with top ends of the fasteners 120 to form states of contact therebetween, respectively, and generate the test signals in response to the states of contact, respectively. After receiving the test signals from the probes 21, respectively, the prompting unit 40 generates the prompting signals based on the test signals, respectively, to confirm the correct installation state and informs the testing worker thereof. In case of at least one empty said installation hole 110, it is impossible for a state of contact to be formed between an affected one of the probes 21 and a missing said fastener 120 which should otherwise be present in the empty installation hole 110; hence, the affected probe 21 does not generate the test signal. Receiving no said test signal from the affected probe 21, the prompting unit 40 informs the testing worker of the incorrect installation state.

Referring to FIG. 4, in the situation where a fastener 120′ is incorrectly installed in an installation hole 110′, for example, when the fastener 120′ is not fully screwed to reach the bottom of the installation hole 110′, the distance between the top end of the fastener 120′ and the tip of a probe 21′ is shorter than the distance between the top end of each of the fasteners 120 and the tip of a corresponding one of the probes 21 when the fasteners 120 are correctly installed in the installation holes 110, respectively. Hence, in the course of driving the testing unit 20 to approach the subject 100 with the mobile platform 30, the probe 21′ comes into contact with the fastener 120′ incorrectly installed in the installation hole 110′ to form a state of contact therebetween earlier than the probes 21 do, and in consequence the probe 21′ generates, earlier than the probes 21 do, the test signal which is then received by the prompting unit 40. As a result, the prompting unit 40 informs the testing worker that the fastener 120′ is correctly installed in the installation hole 110′ and that the installation holes 110 are not correctly installed with the fasteners 120, respectively. As described above, what the prompting unit 40 informs the testing worker of is misleading seemingly but revealing indeed. Being informed that installation holes correctly installed with fasteners are few or even unique; the testing worker can immediately single out the allegedly-good installation hole(s) for reexamination. In doing so, the testing worker is able to determine quickly that the installation hole 110′ is not correctly installed with the fastener 120′.

Regarding the fastener testing device in this embodiment, the prompting unit 40 comprises a plurality of light-emitting elements 41. The light-emitting elements 41 correspond in position to the installation holes 110, respectively, such that the light-emitting elements 41 and the installation holes 110 have a one-to-one relationship conducive to indicating whether the installation holes 110 are correctly installed with the fasteners 120, respectively. Hence, the prompting signals of the light-emitting elements 41 are effective in identifying any said installation holes 110 not correctly installed with the fasteners 120. The light-emitting elements 41 are light-emitting diodes but not necessarily so; on the contrary, any light-emitting elements capable of functioning as indicators are applicable to the present invention.

Regarding the fastener testing device in this embodiment, the prompting unit 40 further comprises an alerting unit 42. The alerting unit 42 generates, according to the prompting signals, a correct installation state alerting signal for indicating the correct installation state and an incorrect installation state alerting signal for indicating the incorrect installation state as appropriate. The alerting unit 42 generates the correct installation state alerting signal if the prompting signals indicate that all the installation holes 110 are correctly installed with the fasteners 120, respectively. The alerting unit 42 generates the incorrect installation state alerting signal if at least one of the prompting signals indicates that one of the installation holes 110 is empty or is not correctly installed with a corresponding one of the fasteners 120. As mentioned above, the alerting unit 42 generates the correct installation state alerting signal and the incorrect installation state alerting signal for indicating the correct installation state and the incorrect installation state, respectively. Alternatively, in a variant embodiment, the alerting unit 42 is capable of generating the correct installation state alerting signal only, to indicate the correct installation state; hence, if the alerting unit 42 does not generate the correct installation state alerting signal, it will mean that the incorrect installation state prevails. Conversely, in another variant embodiment, the alerting unit 42 is capable of generating the incorrect installation state alerting signal only, to indicate the incorrect installation state; hence, if the alerting unit 42 does not generate the incorrect installation state alerting signal, it will mean that the correct installation state prevails. The alerting unit 42 is a buzzer capable of producing different sounds for indicating the correct installation state and the incorrect installation state, respectively. In a variant embodiment, the buzzer is capable of producing the same sound only, wherein the buzzer buzzes to indicate the correct installation state and keeps quiet to indicate the incorrect installation state or, alternatively, the buzzer buzzes to indicate the incorrect installation state and keeps quiet to indicate the correct installation state. In another variant embodiment, the alerting unit 42 comprises two buzzers each capable of producing a distinctive sound, and thus the two different sounds serve to indicate the correct installation state and the incorrect installation state, respectively. Furthermore, the alerting unit 42 can be a light alerting element which comprises two light-emitting diodes of two different colors, respectively, for indicating the correct installation state and the incorrect installation state, respectively. In a variant embodiment, the light alerting element comes in the form of a single light-emitting diode that is on to indicate the correct installation state and off to indicate the incorrect installation state; alternatively, the light-emitting diode is on to indicate the incorrect installation state and off to indicate the correct installation state.

Regarding the fastener testing device in this embodiment, the testing unit 20 further comprises an adjusting portion 22 for adjusting the arrangement of the probes 21 to the shape of the subject 100 or the depth of the installation holes 110, such that the probes 21 can come into contact with the top ends of the fasteners 120 to form states of contact therebetween, respectively. Referring to FIG. 5, the adjusting portion 22 is a cylindrical body having an installation aperture (not shown). The installation apertures correspond in position to the installation holes 110 of the subject 100 for installing the probes 21, respectively. Varying the depth of the installation apertures is a way of adjusting the length of the probes 21 protruding from the cylindrical body. Alternatively, the length of the probes 21 protruding from the cylindrical body can be adjusted by varying the length of the threads in the installation apertures. The purpose of adjusting the length of the probes 21 protruding from the cylindrical body is to allow the probes 21 to correspond in position to the installation holes 110, respectively, and come into contact with the top ends of the fasteners 120 to form states of contact therebetween, respectively.

Referring to FIG. 6A and FIG. 6B, the probes 21 each further comprise a triggering portion 211 and a retractable probe 212. The triggering portion 211 is electrically connected to the prompting unit (not shown). The retractable probe is retractably disposed at the tip of each of the probes 21. The triggering portion 211 is spaced apart from the retractable probe 212 by a preset distance D. After coining into contact with a corresponding one of the fasteners 120, the retractable probe 212 travels the preset distance D in the direction of the triggering portion 211 to reach the triggering portion 211 and thereby get electrically connected thereto, thus forming a conductive loop and generating the test signal. The conductive loop is formed according to the circuit design of a circuit board (not shown) mounted on the testing unit 20 and electrically connected to the prompting unit 40.

As shown in FIG. 1 through FIG. 3, the mobile platform 30 comprises a handle 31, a connecting rod 32 connected to the handle 31, and a lifting table 33 connected to the connecting rod 32. The testing unit 20 is disposed beneath the lifting table 33 and faces the receiving space 11. The handle 31 guides the connecting rod 32 and drives the lifting table 33 to ascend or descend. To operate the fastener testing device, the testing worker grips the handle 31 and lowers the lifting table 33 such that the testing unit 20 approaches the subject 100 in the receiving space 11 until the probes 21 are aligned with or admitted into the installation holes 110, respectively, for examination.

Referring to FIG. 3, the fastener testing device in this embodiment further comprises a mobile carrier 50 for carrying the subject 100. A sliding rail 12 is disposed on the base 10. A guiding groove 52 is disposed beneath the mobile carrier 50. The sliding rail 12 and the guiding groove 52 correspond in position to each other and are engageable with each other, thereby allowing the mobile carrier 50 to enter and exit the receiving space 11. The testing worker mounts the subject 100 on the mobile carrier 50 and then moves the mobile carrier 50 into the receiving space 11 by sliding the sliding rail 12 along the guiding groove 52, and in consequence the testing worker has the subject 100 received in the receiving space 11 conveniently.

Accordingly, the fastener testing device of the present invention is used in examining an installation state of engagement between installation holes of a subject and fasteners, respectively, in a quick, convenient, and precise manner. The fastener testing device indicates how well each of the fasteners is installed in a corresponding one of the installation holes, so as to identify incorrect fastener-installed ones or empty ones of the installation holes of the subject. The fastener testing device generates an alerting signal for indicating that all the installation holes are correctly installed with the fasteners, respectively, and generates another alerting signal for indicating that one of the installation holes is empty or is not correctly installed with a corresponding one of the fasteners. The fastener testing device adjusts the manner of presentation of the testing unit to the height or position of each of the fasteners inserted into the installation holes of the subject, such that the installation state of engagement between the installation holes and the fasteners, respectively, can be examined in a single process. As a result, the fastener testing device of the present invention is not prone to negligence or manmade errors but is time-saving and labor-saving, thereby cutting costs.

The present invention is disclosed above by a preferred embodiment. However, persons skilled in the art should understand that the preferred embodiment is illustrative of the present invention only, but should not be interpreted as restrictive of the scope of the present invention. Hence, all equivalent modifications and replacements made to the aforesaid embodiment should fall within the scope of the present invention. Accordingly, the legal protection for the present invention should be defined by the appended claims.

What is claimed is:

1. A fastener testing device for examining an installation state of engagement between a plurality of installation holes of a subject and a plurality of fasteners, respectively, the fastener testing device comprising:
a base defined with a receiving space thereabove for receiving the subject therein;
a testing unit comprising a plurality of probes corresponding in position to the installation holes, respectively;
a mobile platform disposed above the base and connected to the testing unit for driving the testing unit to move relative to the receiving space, such that the testing unit generates a test signal in response to a state of contact between each of the probes and a corresponding one of the fasteners; and
a prompting unit electrically connected to the testing unit for generating prompting signals based on the test signals, respectively.

2. The fastener testing device of claim 1, wherein the prompting unit comprises a plurality of light-emitting elements corresponding in position to the installation holes, respectively, for indicating the installation state of each of the fasteners in the installation holes, respectively.

3. The fastener testing device of claim 2, wherein the light-emitting elements are light-emitting diodes.

4. The fastener testing device of claim 1, wherein the prompting unit further comprises an alerting unit for generating, based on the prompting signals, at least one of a correct installation state alerting signal for indicating a correct installation state and an incorrect installation state alerting signal for indicating an incorrect installation state.

5. The fastener testing device of claim 4, wherein the alerting unit is one of a buzzer and a light alerting element.

6. The fastener testing device of claim 1, wherein the testing unit further comprises an adjusting portion for adjusting a manner of presentation of the probes to a shape of the subject or to a depth of the installation holes.

7. The fastener testing device of claim 1, wherein the probes each further comprise a triggering portion and a retractable probe, the triggering portion being electrically connected to the prompting unit and spaced apart from the retractable probe by a preset distance, wherein, after coming into contact with a corresponding one of the fasteners, the retractable probe travels the preset distance toward the triggering portion to get electrically connected thereto, thus generating the test signal.

8. The fastener testing device of claim 1, wherein the mobile platform comprises a handle, a connecting rod connected to the handle, and a lifting table connected to the connecting rod, the handle guiding the connecting rod and driving the lifting table to ascend or descend, wherein the testing unit is disposed beneath the lifting table.

9. The fastener testing device of claim 1, further comprising a mobile carrier for carrying the subject, wherein the mobile carrier and the base have a guiding groove and a sliding rail, respectively, thereby allowing the mobile carrier to enter and exit the receiving space.

10. The fastener testing device of claim 1, wherein the fasteners and the installation holes are threaded correspondingly.
